(19) United States
(12) Patent Application Publication
(10) Pub. No.: US 2023/0162356 A1
(43) Pub. Date: May 25, 2023

(54) DIAGNOSTIC IMAGING DEVICE, DIAGNOSTIC IMAGING METHOD, DIAGNOSTIC IMAGING PROGRAM, AND LEARNED MODEL

(71) Applicants: JAPANESE FOUNDATION FOR CANCER RESEARCH, Tokyo (JP); AI Medical Service Inc., Tokyo (JP)

(72) Inventors: Yusuke HORIUCHI, Tokyo (JP); Tomohiro TADA, Tokyo (JP)

(21) Appl. No.: 17/995,592

(22) PCT Filed: Apr. 9, 2021
(86) PCT No.: PCT/JP2021/015061

§ 371 Date: Oct. 6, 2022

(30) Foreign Application Priority Data
Apr. 10, 2020 (JP) 2020-070848

(51) Int. Cl. G06T 7/00; G06T 7/70; G06T 11/00; A61B 1/00; A61B 1/06

(52) U.S. Cl. CPC G06T 7/0012; G06T 7/70; G06T 11/00; A61B 1/000096; A61B 1/0005; A61B 1/00055; A61B 1/063; G06T 2207/10068; G06T 2207/30096; G06T 2207/10016; G06T 2207/20081; G06T 2207/20084; G06T 2207/30092; G06T 2210/41

(57) ABSTRACT

Provided are a diagnostic imaging device, diagnostic imaging method, diagnostic imaging program, and learned model with which gastric cancer diagnosis can be carried out in real time during endoscopic examination performed using NBI in combination with a magnifying endoscope. The diagnostic imaging device comprises an endoscopic video image acquisition unit which emits narrow-band light at a subject's stomach and acquires an endoscopic video image captured while the stomach is in a state of magnified observation, and an estimation unit which uses a convolutional neural network, which has been caused to learn using gastric cancer images and non-gastric cancer images as training data, to estimate the presence of gastric cancer in the acquired endoscopic video image, and outputs estimation results.

### TECHNICAL FIELD

The present invention relates to an image diagnosis apparatus, an image diagnosis method, an image diagnosis program and a learned model.

### BACKGROUND ART

Gastric cancer is one of the most common cancers recognized in the world and has one of the highest cancer-related mortality rates. On the other hand, with the development of endoscopic instruments, gastric cancer is increasingly being detected at an early stage by endoscopy. As a result, the mortality rate from gastric cancer has been decreasing in recent years. Furthermore, with the development of endoscopic submucosal dissection (ESD), treatment of early-stage gastric cancer has become a minimally invasive procedure. However, according to Japanese guidelines for the treatment of gastric cancer, the indication for ESD is limited to intramucosal cancer (cancer that has invaded down to the mucosal intrinsic layer), and it is important to detect and diagnose gastric cancer at an earlier stage.

In general, the diagnosis of gastric cancer is made by endoscopy. Recently, a magnifying endoscope with Narrow Band Imaging (ME-NBI) has been developed, which enables magnified observation of the stomach by irradiating a narrow band of light (NBI: Narrow Band Imaging) on the stomach of a subject. It has been reported that the magnifying endoscope with NBI has a higher diagnostic performance for gastric cancer than conventional endoscopes. However, endoscopists need to make considerable efforts to master the diagnostic techniques of gastric cancer using ME-NBI. This is because it is difficult to distinguish between gastric cancer and gastritis, since most gastric cancers have chronic inflammation (gastritis) associated with H. pylori infection in the background mucosa. Especially in cases with strong inflammatory cell infiltration, the localization and extent of gastric cancer may be unclear, and inexperienced endoscopists tend to miss gastric cancer. Therefore, more advanced diagnostic techniques are required of endoscopists. Therefore, it is more difficult to properly diagnose gastric cancer compared to other gastrointestinal cancers in which chronic inflammation associated with H. pylori infection is not observed in the background mucosa (e.g., esophageal cancer, which is judged by the color and irregularity of the mucosa, and colon cancer, which is characterized by polyps).

In recent years, artificial intelligence (AI) using deep learning has been developed and applied in the medical field. Convolutional Neural Network (CNN), which performs convolutional learning while maintaining the features of images input to AI, has been developed, dramatically improving the image diagnostic capability of computer-aided diagnosis (CAD) systems that classify learned images.

AI using deep learning has attracted attention in various medical fields, including radiation oncology, skin cancer classification, diabetic retinopathy, histological classification of gastric biopsies, and characterization of colorectal lesions using hyper-magnifying endoscope. In particular, it has been proven that AI can achieve the same accuracy as a specialist at the microscopic endoscopy level (see NPL 1). In dermatology, it has also been published that AI with deep learning capabilities can produce diagnostic imaging capabilities equivalent to those of specialists (see NPL 2), and patent literature using various machine learning methods (see PTLS 1 and 2) also exist.

However, when still images are used as training data for training, and the AI makes judgments based on still images taken during the examination, the AI cannot make judgments unless still images are taken, so it is necessary to keep in mind that it should be noted that AI cannot assist in determining whether or not a lesion is missed during endoscopy. In addition, when judging as a video in real time, it is considered beneficial in actual clinical practice in terms of increasing the number of cancers to be detected, since it assists in the detection of cancers during endoscopy.

### CITATION LIST

### Patent Literature

### PTL 1

### Japanese Patent Application Laid-Open No. 2017-045341

### PTL 2

### Japanese Patent Application Laid-Open No. 2017-067489

### Non-Patent Literature

### NPL 1

http://www.giejournal.org/article/S0016-5107(14)02171-3/fulltext, “Novel computer-aided diagnostic system for colorectal lesions by using endocytoscopy” Yuichi Mori et. al. Presented at Digestive Disease Week 2014, May 3-6, 2014, Chicago, Ill., USA

### NPL 2

Nature, February 2017, Volume 1, Article, “Learning about skin lesions: enhancing the ability of artificial intelligence to detect skin cancer from images.” (http://www.natureasia.com/ja-jp/nature/highlights/82762)

### NPL 3

Horiuchi Y, Aoyama K, Tokai Y, et al. Convolutional neural network for differentiating gastric cancer from gastritis using magnified endoscopy with narrow band imaging. Dig Dis Sci. 2019. doi: 10.1007/s10620-019-05862-6.

### NPL 4

Li L, Chen Y, Shen Z, et al. Convolutional neural network for the diagnosis of early gastric cancer based on magnifying narrow band imaging. Gastric Cancer. 2019; 23(1):126-132. doi:10.1007/s10120-019-00992-2.10.1007/s10120-019-00992-2.

### NPL 5

Ishioka M, Hirasawa T, Tada T. Detecting gastric cancer from video images using convolutional neural networks. Dig Endosc. 2019; 31(2):e34—e35. doi: 10.1111/den.13306.

### SUMMARY OF INVENTION

### Technical Problem

As described above, it has been suggested that the diagnostic imaging capability of AI is comparable to that of a specialist endoscopist. However, in gastric endoscopy using a magnifying endoscope with NBI, the diagnostic imaging technology that uses AI's diagnostic imaging capability to diagnose gastric cancer in real time has not yet been introduced in actual medical practice (real clinical practice). In addition, the technology has not yet been introduced in actual medical practice (actual clinical practice), and is expected to be put to practical use in the future. Meanwhile, for the diagnosis of digestive cancers using endoscopy, it is important to design AI programs in line with the characteristics of each cancer type, since the extraction of unique features of each digestive cancer (esophageal, gastric, colorectal, etc.) and the determination of its pathological level are different.

An object of the present invention is to provide an image diagnosis apparatus, an image diagnosis method, an image diagnosis program and a learned model that can perform the diagnosis of gastric cancer in real time in gastrointestinal endoscopy using an NBI combined magnifying endoscope.

### Solution to Problem

An image diagnosis apparatus according to the present invention includes: an endoscopic video acquisition section configured to acquire an endoscope video captured in a state where a stomach of a subject is irradiated with narrowband light and the stomach is observed in a magnified manner; and an estimation section configured to estimate the presence of a gastric cancer in the acquired endoscope video by using a convolutional neural network, and output an estimation result, the convolutional neural network having been subjected to learning with a gastric cancer image and a non-gastric cancer image as training data.

An image diagnosis method according to the present invention includes: acquiring an endoscope video captured in a state where a stomach of a subject is irradiated with narrowband light and the stomach is observed in a magnified manner; and estimating the presence of a gastric cancer in the acquired endoscope video by using a convolutional neural network, and outputting an estimation result, the convolutional neural network having been subjected to learning with a gastric cancer image and a non-gastric cancer image as training data.

An image diagnosis program according to the present invention is configured to cause a program to execute: an endoscopic video acquisition process of acquiring an endoscope video captured in a state where a stomach of a subject is irradiated with narrowband light and the stomach is observed in a magnified manner; and an estimation process of estimating the presence of a gastric cancer in the acquired endoscope video by using a convolutional neural network, and outputting an estimation result, the convolutional neural network having been subjected to learning with a gastric cancer image and a non-gastric cancer image as training data.

A learned model according to the present invention is obtained through learning of a convolutional neural network with a gastric cancer image and a non-gastric cancer image as training data, the learned model being configured to cause a computer to estimate the presence of a gastric cancer in an endoscope video captured in a state where a stomach of a subject is irradiated with narrowband light and the stomach is observed in a magnified manner, and output an estimation result.

### Advantageous Effects of Invention

According to the present invention, in gastrointestinal endoscopy using an NBI combined magnifying endoscope, the diagnosis of gastric cancer in real time can be performed.

### BRIEF DESCRIPTION OF DRAWINGS

FIG. 1 is a block diagram illustrating a general configuration of an image diagnosis apparatus of the present embodiment;

FIG. 2 is a diagram illustrating a hardware configuration of the image diagnosis apparatus of the present embodiment;

FIG. 3 is a diagram illustrating an architecture of a convolutional neural network of the present embodiment;

FIGS. 4A and 4B are diagrams illustrating an example where determination result images are displayed in a superimposed manner on endoscope videos in the present embodiment;

FIGS. 5A to 5C are diagrams illustrating an example of an endoscopic image used as training data;

FIG. 6 is a diagram illustrating features of a subject and lesion (gastric cancer) related to an endoscope video used for an evaluation test data set;

FIG. 7 is an ROC curve with values of a predetermined time, degree of certainty and predetermined number with which the correct diagnosis rate of the image diagnosis apparatus is highest;

FIG. 8 is a diagram illustrating the numbers of correct diagnoses, false diagnoses and undetermined diagnoses of the image diagnosis apparatus and 11 skilled endoscopists for an endoscope video where gastric cancer is present and an endoscope video where gastric cancer is not present; and

FIG. 9 is a diagram illustrating correct diagnosis rates, sensitivities, specificities, positive predictive values and negative predictive values of the image diagnosis apparatus and 11 skilled endoscopists.

### DESCRIPTION OF EMBODIMENTS

The present embodiments are described in detail below with reference to the drawings.

### General Configuration of Image Diagnosis Apparatus

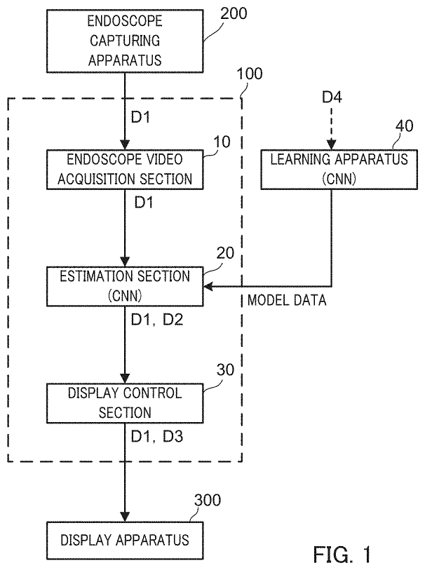

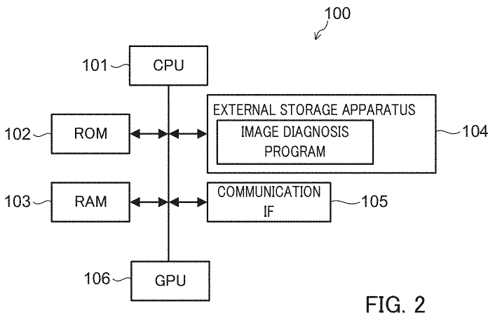

First, a configuration of image diagnosis apparatus 100 of the present embodiment is described. FIG. 1 is a block diagram illustrating a general configuration of image diagnosis apparatus 100. FIG. 2 is a diagram illustrating an example of a hardware configuration of image diagnosis apparatus 100 of the present embodiment.

In endoscopy of a digestive organ (in the present embodiment, stomach) conducted by a doctor (for example, an endoscopist), image diagnosis apparatus 100 performs diagnosis of gastric cancer in real time by use of the image diagnostic capability for the endoscopic image of a convolutional neural network (CNN). Image diagnosis apparatus 100 is connected with endoscope capturing apparatus 200 and display apparatus 300.

Endoscope capturing apparatus 200 is an electronic endoscope (also referred to as video scope) with a built-in image-capturing means, a camera-equipped endoscope including an optical endoscope in which a camera head with a built-in image-capturing means is mounted or the like, for example. Endoscope capturing apparatus 200 is inserted to a digestive organ from the mouse or nose of the subject so as to capture an image of the diagnostic target portion in the digestive organ, for example.

In the present embodiment, endoscope capturing apparatus 200 captures the diagnostic target portion in the stomach in the form of an endoscope video in accordance with the operation (for example, button operation) of the doctor, in the state where the stomach of the subject is irradiated with narrowband light (for example, NBI narrowband light) and the stomach is magnified 80 times, for example. The endoscope video is composed of a plurality of temporally sequential endoscopic images. Endoscope capturing apparatus 200 outputs endoscopic video data D1 representing the captured endoscope video to image diagnosis apparatus 100.

Display apparatus 300 is, for example, a liquid crystal display, and identifiably displays, to the doctor, the determination result image and the endoscope video output from image diagnosis apparatus 100.

As illustrated in FIG. 2, image diagnosis apparatus 100 is a computer including, as main components, central processing unit (CPU) 101, read only memory (ROM) 102, random access memory (RAM) 103, external storage apparatus (for example, flash memory) 104, communication interface 105 and graphics processing unit (GPU) 106 and the like.

Each function of image diagnosis apparatus 100 is implemented with reference to the control program (such as image diagnosis program) and various data (such as endoscopic video data, learning training data, and the model data (such as structure data and learned weight parameter) of the convolutional neural network stored in CPU 101, GPU 106 ROM 102, RAM 103, external storage apparatus 104 and the like, for example. Note that RAM 103 functions as a working area and a temporary storage area of data, for example.

Note that a part or all of each function of image diagnosis apparatus 100 may be achieved through a process of a digital signal processor (DSP) instead of or together with the processes of CPU 101 and GPU 106. In addition, likewise, a part or all of each function may be achieved through a process of a dedicated hardware circuit instead of or together with the process of software.

As illustrated in FIG. 1, image diagnosis apparatus 100 includes endoscopic video acquisition section 10, estimation section 20 and display control section 30. Learning apparatus 40 has a function of generating the model data (corresponding to “learned model” of the present invention) of the convolutional neural network to be used in image diagnosis apparatus 100. Note that display control section 30 also functions as the “alert output control section” of the present invention.

### Endoscopic Video Acquisition Section

Endoscopic video acquisition section 10 acquires endoscopic video data D1 output from endoscope capturing apparatus 200. Then, endoscopic video acquisition section 10 outputs the acquired endoscopic video data D1 to estimation section 20. Note that when acquiring endoscopic video data D1, endoscopic video acquisition section 10 may directly acquire it from endoscope capturing apparatus 200, or may acquire endoscopic video data D1 stored in external storage apparatus 104 or endoscopic video data D1 provided through Internet connection or the like.

### Estimation Section

With convolutional neural network, estimation section 20 estimates the presence of the lesion (in the present embodiment, gastric cancer) in the endoscope video represented by endoscopic video data D1 output from endoscopic video acquisition section 10, and outputs the estimation result. To be more specific, estimation section 20 estimates the lesion name (name) and lesion location (position) of the lesion present in the endoscope video, and the degree of certainty (also referred to as likelihood) of the lesion name and lesion location. Then, estimation section 20 outputs, to display control section 30, endoscopic video data D1 output from endoscopic video acquisition section 10 and estimation result data D2 representing the estimation results of the lesion name, lesion location and the degree of certainty.

In addition, when a predetermined number (for example, three) of endoscopic images whose degree of certainty is equal to or greater than a predetermined value (for example, 0.5) is sequentially present in a predetermined time (for example, 0.5 seconds) in the endoscope video represented by endoscopic video data D1, estimation section 20 estimates that there is a lesion (gastric cancer) in the endoscope video. Here, the above-mentioned predetermined number is set to be greater as the predetermined value is smaller. When it is estimated that the lesion is present in the endoscope video, estimation section 20 outputs the estimation (estimation result) to display control section 30.

In the present embodiment, estimation section 20 estimates a probability score as an indicator representing the degree of certainty of the lesion name and lesion location. The probability score is represented as a value that is greater than 0 and is equal to or smaller than 1. The higher the probability score is, the higher the degree of certainty of the lesion name and lesion location is.

Note that the probability score is an example of an indicator representing the degree of certainty of the lesion name and lesion location, and any other indicators may be used. For example, the probability score may be represented by values from 0% to 100%, or by a value of multiple-level values.

The convolutional neural network is a kind of feedforward neural network, and is based on the knowledge of the structure of the visual cortex of the brain. Basically, it has a structure in which a convolutional layer responsible for extracting local features of image and a pooling layer (subsampling layer) for collecting features for each locality are repeated. With each layer of the convolutional neural network, a plurality of neurons is provided, and each neuron is disposed in a manner corresponding to the visual cortex. The basic function of each neuron is composed of input of and output of signals. It should be noted that, when transmitting signals to each other, the neurons of each layer do not input the signal as it is, but sets a coupling weight to each input and outputs the signal to the neuron of the next layer when the sum of the weighted inputs exceeds the threshold value set in each neuron. The coupling weights of the neurons are calculated in advance from the learning data. In this manner, the output value can be estimated by inputting real time data. The algorithm making up the network is not limited as long as the convolutional neural network can achieve the object.

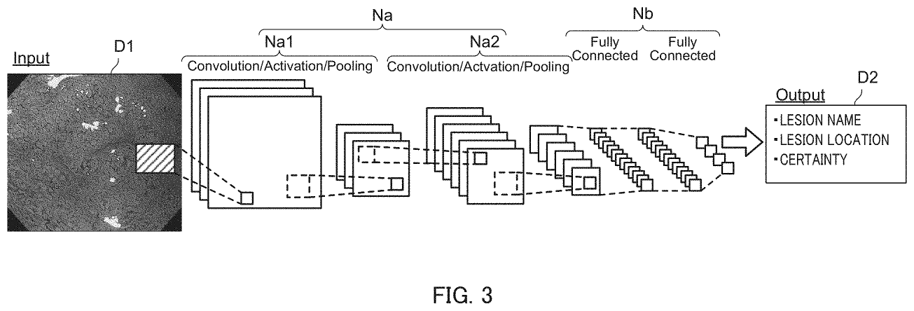

FIG. 3 is a diagram illustrating an architecture of the convolutional neural network of the present embodiment. Note that the model data (such as structure data and learned weight parameter) of the convolutional neural network is stored in external storage apparatus 104 together with an image diagnosis program.

As illustrated in FIG. 3, the convolutional neural network includes feature extraction section Na and identification section Nb, for example. Feature extraction section Na performs a process of extracting the image feature from the input image (more specifically, the endoscopic image making up the endoscope video represented by endoscopic video data D1). Identification section Nb outputs the estimation result of the image from the image feature extracted by feature extraction section Na.

Feature extraction section Na is composed of a plurality of features extraction layers Na1, Na2 . . . hierarchically connected with each other. Each of feature extraction layers Na1, Na2 . . . includes a convolutional layer, an activation layer and a pooling layer.

Feature extraction layer Na1 as the first layer scans the input image in a unit of predetermined sizes through raster scan. Then, feature extraction layer Na1 extracts the feature included in the input image by performing the feature extraction process on the scanned data with the convolutional layer, the activation layer and the pooling layer. Feature extraction layer Na1 as the first layer extracts relatively simple single features such as a linear feature extending in the horizontal direction and a linear feature extending in an oblique direction, for example.

Feature extraction layer Na2 as the second layer scans an image (also called feature map) input from feature extraction layer Na1 of the previous layer in a unit of predetermined sizes through raster scan, for example. Then, feature extraction layer Na2 extracts the feature included in the input image by performing the feature extraction process on the scanned data in the same manner, with the convolutional layer, the activation layer and the pooling layer. Note that feature extraction layer Na2 as the second layer extracts a composite feature of a higher level by performing integration with reference to the positional relationship of the plurality of features extracted by feature extraction layer Na1 as the first layer and the like.

The second and subsequent feature extraction layers (FIG. 3 illustrates only two layers of feature extraction layer Na for convenience of description) execute the process as that of feature extraction layer Na2 as the second layer. Then, the output (the values of the maps of the plurality of feature maps) of the final feature extraction layer is input to identification section Nb.

Identification section Nb is composed of a multilayer perceptron with a plurality of fully connected layers hierarchically connected, for example.

The input side fully connected layer of identification section Nb, which is fully connected to the values of the maps of the plurality of feature maps acquired from feature extraction section Na, performs sum-of-product computation on the values while changing the weight coefficient, and outputs it.

The fully connected layer of the next layer of identification section Nb, which is fully connected to the values output by elements of the fully connected layer of the previous layer, performs sum-of-product computation while applying different weight coefficients to the values. Then, at the last of identification section Nb, a layer (such as softmax function) for outputting the lesion name and lesion location of the lesion present in the image (endoscopic image) input to feature extraction section Na and the probability score (degree of certainty) of the lesion name and lesion location is provided.

The convolutional neural network may have an estimation function such that a desired estimation result (here, lesion name, lesion location and probability score) can be output from the input endoscopic image through a preliminary learning process using reference data (hereinafter referred to as “training data”) preliminarily subjected to a marking process by an experienced endoscopist. At this time, through the learning with a sufficient amount of training data covering typical pathological conditions and proper adjustment of weights, it is possible to prevent overfitting and produce an AI program with generalized capability for gastric cancer diagnosis.

The convolutional neural network of the present embodiment is configured such that, with endoscopic video data D1 as an input (Input of FIG. 3), the lesion name, lesion location and probability score corresponding to the image feature of the endoscopic image making up the endoscope video represented by endoscopic video data D1 are output (Output of FIG. 3) as estimation result data D2.

Note that more preferably, the convolutional neural network may be configured to be able to input information on the age, gender, region, or past medical history of the subject (for example, may be provided as an input element of identification section Nb) in addition to endoscopic video data D1. Since the importance of the real-world data in the actual clinical practice is particularly recognized, addition of the information on the subject attributes can achieve loading in more useful systems in the actual clinical practice. Specifically, the feature of endoscopic image is considered to have correlations with the information on the age, gender, region, past medical history, family medical history and the like of the subject, and therefore, with reference to the subject's property such as the age in addition to endoscopic video data D1 for the convolutional neural network, it is possible to estimate the lesion name and lesion location with higher accuracy. This approach is a matter that should be incorporated, especially if the invention is to be utilized internationally, as the pathological condition of disease can vary by region and even between races.

In addition, estimation section 20 may perform, in addition to the process of the convolutional neural network, a process of conversion to the size and aspect ratio of the endoscopic image, a color division process of the endoscopic image, a color conversion process of the endoscopic image, a color extraction process, a luminance grade extraction process and the like as preprocessing. To prevent overfitting and increase accuracy, it is also preferable to adjust the weighting.

### Display Control Section

Display control section 30 generates a determination result image for superimposition display of the lesion name, lesion location and probability score represented by estimation result data D2 output from estimation section 20 on the endoscope video represented by endoscopic video data D1 output from estimation section 20. Then, display control section 30 outputs endoscopic video data D1 and determination result image data D3 representing the generated determination result image to display apparatus 300. In this case, digital image processing systems for image structure enhancement, color enhancement, differential processing, high contrast and high definition of the lesion of the endoscope video structure may be connected to perform processing for assisting the understanding and determination of the viewer (for example, the doctor).

Display apparatus 300 displays the determination result image represented by determination result image data D3 in a superimposed manner on the endoscope video represented by endoscopic video data D1 output from display control section 30. The endoscope video and determination result image displayed on display apparatus 300 is used for real time diagnosis assistance and diagnosis support for the doctor.

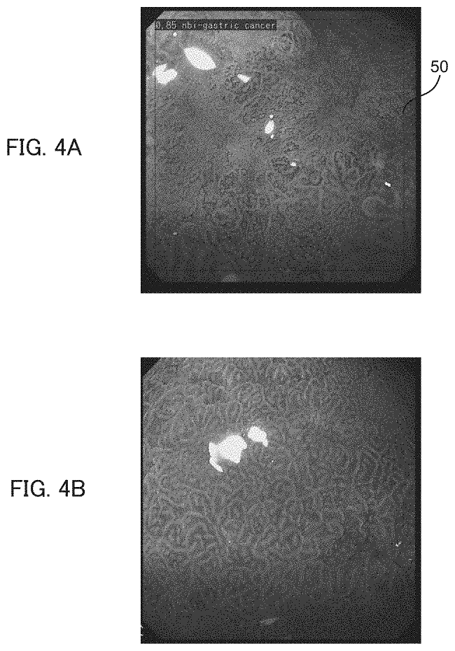

FIG. 4 is a diagram illustrating an example in which a determination result image is displayed in a superimposed manner on an endoscope video. As illustrated in FIG. 4A, as a determination result image, rectangular frame 50 representing the lesion location (range) estimated by estimation section 20, the lesion name (for example, gastric cancer) and the probability score (for example, 0.85) are displayed.

In the present embodiment, when the probability score is equal to or greater than a certain threshold value (for example, 0.4), display control section 30 displays a rectangular frame representing the lesion location, the lesion name and the probability score in a superimposed manner on the endoscope video (see FIG. 4A). On the other hand, when the probability score is smaller than a certain threshold value (for example, 0.4), i.e., when the probability of the presence of a lesion in the endoscope video is low, display control section 30 does not display the rectangular frame representing the lesion location, the lesion name and the probability score on the endoscope video (see FIG. 4B). That is, display control section 30 changes the display mode of the determination result image on the endoscope video in accordance with the probability score represented by estimation result data D2 output from estimation section 20.

In addition, when the estimation that the lesion is present in the endoscope video is output from estimation section 20, display control section 30 controls display apparatus 300 so as to display and output an alert by turning on the light of the display screen of the endoscope video and blinking the rectangular range of the lesion determination section. This effectively draws the attention of the doctor to the presence of the lesion in the endoscope video. Note that when estimation section 20 estimates that the lesion is present in the endoscope video, an alert may be output by sounding (outputting) an alert sound from a speaker not illustrated in the drawing. Further, at this time, the determination probability and estimation probability may be individually calculated and displayed.

### Learning Apparatus

Learning apparatus 40 performs a learning process for the convolutional neural network of learning apparatus 40 by inputting training data D4 stored in an external storage apparatus not illustrated in the drawing such that the convolutional neural network of estimation section 20 can estimate the lesion location, lesion name and probability score from endoscopic video data D1 (more specifically, the endoscopic image making up the endoscope video).

In the present embodiment, learning apparatus 40 performs a learning process by using, as training data D4, an endoscopic image (still picture image) captured with endoscope capturing apparatus 200 through irradiation of a plurality of the stomachs of the subject with narrowband light and magnification of the stomachs in a previously performed gastrointestinal endoscopy, and the lesion name and lesion location of a lesion (gastric cancer) present in the endoscopic image determined in advance by a doctor. To be more specific, learning apparatus 40 performs the learning process of the convolutional neural network such that errors (also called loss) of the output data for the correct value (lesion name and lesion location) obtained when the endoscopic image is input to the convolutional neural network are reduced.

In the present embodiment, learning apparatus 40 performs a learning process by using, as training data D4, an endoscopic image (corresponding to “gastric cancer image” of the present invention) in which the lesion (gastric cancer) is shown, i.e., present and an endoscopic image (corresponding to “non-gastric cancer image” of the present invention) in which the lesion (gastric cancer) is not shown, i.e., not present.

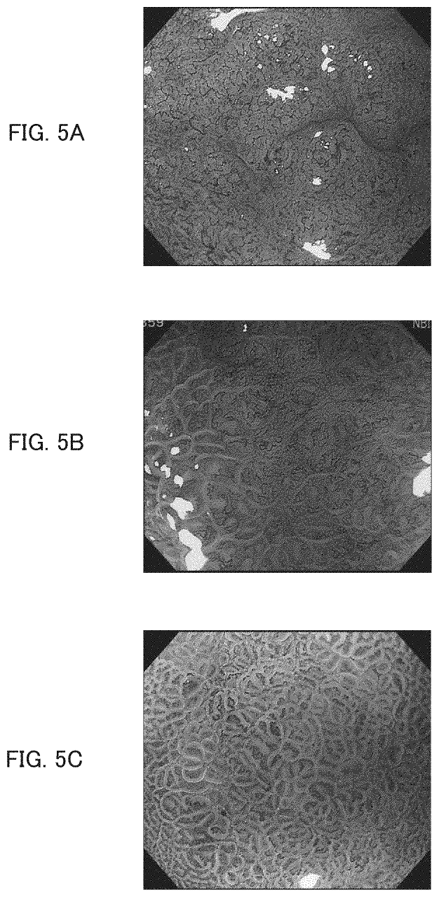

FIG. 5 is diagrams illustrating an example of an endoscopic image used as training data. FIG. 5A illustrates an endoscopic image (gastric cancer image) in which a differentiated gastric cancer as a lesion is present. FIG. 5B illustrates an endoscopic image (gastric cancer image) in which an undifferentiated gastric cancer as a lesion is present. FIG. 5C illustrates an endoscopic image (non-gastric cancer image) in which no gastric cancer as a lesion is not present.

For the endoscopic image as training data D4 in the learning process, the extensive database of Japan's top-class hospital specializing in cancer treatment was mainly used, and marking of the lesion location of the lesion (gastric cancer) was performed through specific examination, sorting, and precise manual processing on all images by a preceptor of Japan Gastroenterological Endoscopy Society with extensive diagnostic and therapeutic experience. For accuracy management and bias elimination of training data D4 (endoscopic image data) serving as reference data, a sufficient number of cases having been subjected to image sorting, lesion identification, and feature extraction marking by expert endoscopists with extensive experience are significantly important because it is directly related to the diagnosis accuracy of image diagnosis apparatus 100. With such highly accurate data cleansing operation and high quality reference data, highly reliable output results of the AI program are provided.

Training data D4 of the endoscopic image may be pixel value data, or data having been subjected to a predetermined color conversion process and the like. In addition, as preprocessing, it is also possible to use the texture feature, the shape feature, the unevenness status, the spreading feature and the like specific to cancerous areas extracted through comparison between an inflammation image and a non-inflammation image. In addition, training data D4 may be associated with information on the age, gender, region, past medical history, and family medical history of the subject and the like, in addition to the endoscopic image data to perform the learning process.

Note that the algorithm for the learning process of learning apparatus 40 may be a publicly known method. Learning apparatus 40 performs a learning process on the convolutional neural network by using, for example, publicly known backpropagation, and adjusts the network parameters (weight coefficient, bias and the like). Then, the model data (such as structure data and learned weight parameter) of the convolutional neural network having been subjected to the learning process with learning apparatus 40 is stored in external storage apparatus 104 together with the image diagnosis program, for example. Examples of the publicly known CNN model include GoogLeNet, ResNet and SENet.

As described in detail above, in the present embodiment, image diagnosis apparatus 100 includes endoscopic video acquisition section 10 that acquires an endoscope video captured in the state where the stomach of the subject is irradiated with narrowband light and the stomach is observed in a magnified manner, and estimation section 20 that estimates the presence of gastric cancer in the acquired the endoscope video by using a convolutional neural network adjusted with a gastric cancer image and a non-gastric cancer image as training data, and outputs the estimation result.

To be more specific, the convolutional neural network has been subjected to learning based on endoscopic images (gastric cancer images and non-gastric cancer images) of a plurality of stomachs (digestive organs) obtained in advance for each of a plurality of subjects, and the definitive determination result of the lesion name and lesion location of the lesion (gastric cancer) obtained in advance for each of a plurality of subjects. Thus, the lesion name and lesion location of the stomach of a new subject can be estimated in short time with the accuracy substantially comparable to that of experienced endoscopists. Thus, in gastrointestinal endoscopy, diagnosis of gastric cancer can be performed in real time by using the diagnostic capability of the endoscope video of the convolutional neural network according to the present embodiment. In the actual clinical practice, image diagnosis apparatus 100 may be used as a diagnosis support tool that directly supports the diagnosis of the endoscope video conducted by an endoscopist in the examination room. In addition, image diagnosis apparatus 100 may be used for a central diagnosis support service that supports the diagnosis of endoscope videos transmitted from a plurality of examination rooms, and for a diagnosis support service that supports the diagnosis of the endoscope video at remote institutions through remote control via Internet connection. In addition, image diagnosis apparatus 100 may be operated on the cloud. Further, these endoscope videos and AI determination results may be provided directly as a video library so as to be used as teaching materials and resources for educational training and research.

The above embodiments are merely examples of embodiments for implementing the invention, and the technical scope of the invention should not be interpreted as limited by them. In other words, the invention can be implemented in various forms without deviating from its gist or its main features.

### Example Experiment

Finally, an evaluation test for confirming the effects of the configuration of the present embodiment is described.

### Preparation of Training Data Set

In cases (395 cases) in which ESD was performed as initial treatment at the Cancer Institute Hospital of JFCR between April 2005 to December 2016, 1492 endoscopic images with gastric cancer captured with an endoscope capturing apparatus in the state where the stomachs of a plurality of subjects are irradiated with narrowband light and the stomachs are observed in a magnified manner, and 1078 endoscopic images with no gastric cancer captured with an endoscope capturing apparatus in the state where the stomachs of a plurality of subjects are irradiated with narrowband light and the stomachs are observed in a magnified manner were extracted from the electronic medical record apparatus and prepared as the training data set (training data) used for the learning of the convolutional neural network in the image diagnosis apparatus. As the endoscope capturing apparatus, GIF-H240Z, GIF-H260Z and GIF-H290 available from Olympus Medical Systems Corp were used.

Note that the endoscopic images as the training data set include endoscopic images captured with an endoscope capturing apparatus in the state where the stomach of the subject is strongly enlarged and observed, and endoscopic images in which gastric cancer is found (present) in 60% or more of the entire image. On the other hand, endoscopic images whose image quality is poor due to mucus and blood adhering in a wide area, out of focus or halation were excluded from the training data set. A Japan Gastroenterological Endoscopy Society preceptor, specialist in gastric cancer, prepared the training data set by specifically examining and sorting the prepared endoscopic images and performing marking of lesion locations through precise manual processing.

### Learning and Algorithm

To construct an image diagnosis apparatus for performing the diagnosis of gastric cancer, GoogleNet composed of 22 layers with a structure common to the previous CNN and a sufficient number of parameters and expressive power was used as a convolutional neural network. Caffedeep learning framework developed at Berkeley Vision and Learning Center (BVLC) was used for the learning and evaluation test. All layers of the convolutional neural network were fine-tuned using stochastic gradient descent with a global learning rate of 0.0001. To provide compatibility with CNN, each endoscopic image was resized to 224×224 pixels.

### Preparation of Evaluation Test Data Set

To evaluate the diagnosis accuracy of the image diagnosis apparatus of the constructed convolutional neural network base, in cases in which ESD was performed as initial treatment at the Cancer Institute Hospital of JFCR between April 2019 to August 2019, 87 endoscope videos with gastric cancer captured with an endoscope capturing apparatus in the state where the stomachs of a plurality of subjects are irradiated with narrowband light and the stomachs are observed in a magnified manner, and 87 endoscope videos with gastric cancer captured with an endoscope capturing apparatus in the state where the stomachs of a plurality of subjects are irradiated with narrowband light and the stomachs are observed in a magnified manner were collected as evaluation test data set. To be more specific, in the same cases, after the periphery of the lesion was marked before ESD, endoscope videos in which gastric cancers are shown and endoscope videos in which gastric cancers are not shown were captured. The frame rate of each endoscope video making up the evaluation test data set is 30 fps (one endoscopic image =0.033 seconds). As the endoscope capturing apparatus, as for the preparation of the training data set, GIF-H240Z, GIF-H260Z and GIF-H290 available from Olympus Medical Systems Corp were used.

Note that the evaluation test data set includes, as the endoscope video that meets eligibility criteria, endoscope videos captured for ten seconds with the endoscope capturing apparatus in the state where the stomach of the subject is strongly enlarged and observed. On the other hand, the endoscope videos whose image quality is poor due to mucus and blood adhering in a wide area, out of focus or halation were excluded from the evaluation test data set as endoscope videos that meets the exclusion criteria. A Japan Gastroenterological Endoscopy Society preceptor, a specialist in gastric cancer, prepared the evaluation test data set by specifically examining the prepared endoscope videos and sorting the endoscope video where the gastric cancer is present and the endoscope video where the gastric cancer is not present.

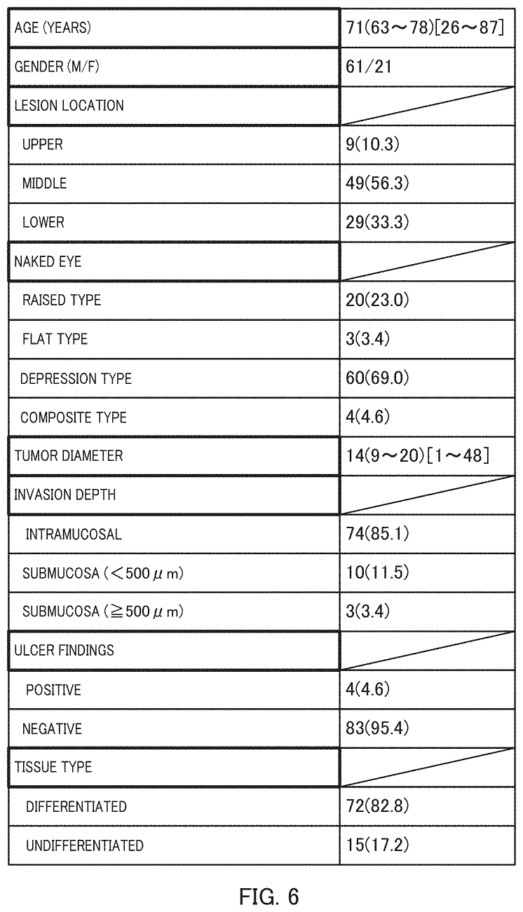

FIG. 6 is a diagram illustrating features of the subject and the lesion (gastric cancer) related to the endoscope video used for the evaluation test data set. In FIG. 6, the numbers in parentheses are the percentage (%) of total. It should be noted that, for the age and the tumor diameter, the center value (quartile range) [entire range] is shown. As illustrated in FIG. 6, for example, the center value of the tumor diameter was 14 mm, and the quartile range (entire range) of the tumor was 9 to 20 (1 to 48) mm. With the naked eye (classification), the depression type was largest, with 60 lesions (69.0%). In terms of the depth of invasion, results were 74 lesions (85.1%) in the intramucosal, ten lesions (11.5%) in the submucosa (<500 μm), and three lesions (3.4%) in the submucosa (≥500 μm).

### Method of Evaluation Test

In the present evaluation test, the evaluation test data set was input to the image diagnosis apparatus of the convolutional neural network base having been subjected to a learning process using the training data set, and whether the presence of the gastric cancer in each endoscope video making up the evaluation test data set can be properly diagnosed was evaluated. The image diagnosis apparatus diagnoses that a lesion is present in the endoscope video when there are a predetermined number of continuous endoscopic images whose degree of certainty is equal to or greater than a predetermined value within a predetermined time. In the present evaluation test, the predetermined time, degree of certainty and the predetermined number were changed to various values, and whether the presence of the gastric cancer in each endoscope video can be properly diagnosed was evaluated by using the values after the change. Then, the values of the predetermined time, the degree of certainty and the predetermined number of the image diagnosis apparatus with which the correct diagnosis rate (described later) is highest was determined, the Receiver Operating Characteristic (ROC) curve thereof was generated, and the area under the curve (AUC) was calculated.

In addition, in the present evaluation test, for the comparison between the diagnostic capability of the image diagnosis apparatus and the diagnostic capability of a skilled endoscopist (specialist) who have mastered the diagnosis technique of gastric cancer of ME-NBI, the skilled endoscopist made a diagnosis as to whether the gastric cancer is present in the endoscope video by viewing each endoscope video making up the evaluation test data set one time. Note that as the skilled endoscopist, 11 Japan Gastroenterological Endoscopy Society certified medical specialists who having conducted the diagnosis of gastric cancer of ME-NBI in the actual clinical practice in the Cancer Institute Hospital of JFCR were selected.

In the present evaluation test, the correct diagnosis rate, sensitivity, specificity, positive predictive value (PPV) and negative predictive value (NPV) with respect to the diagnostic capability of the image diagnosis apparatus (or skilled endoscopist) were calculated by using the following expressions (1) to (5).

Correct diagnosis rate=(the number of endoscope videos where the presence of gastric cancer was properly diagnosed in the evaluation test data set)/(the number of all endoscope videos making up the evaluation test data set) (1)

Sensitivity=(the number of endoscope videos where the presence of gastric cancer was properly diagnosed in the evaluation test data set)/(the number of endoscope videos where the gastric cancer is actually present in the evaluation test data set) (2)

Specificity=(the number of endoscope videos where the non-presence of gastric cancer was properly diagnosed in the evaluation test data set)/(the number of endoscope videos where the gastric cancer is actually not present in the evaluation test data set) (3)

Positive predictive value (PPV)=(the number of endoscope videos where the gastric cancer is actually present among endoscope videos diagnosed that the gastric cancer is present in the evaluation test data set)/(the number of endoscope videos diagnosed that the gastric cancer is present in the evaluation test data set) (4)

Negative predictive value (NPV)=(the number of endoscope videos where the gastric cancer is actually not present among endoscope videos diagnosed that the gastric cancer is not present in the evaluation test data set)/(the number of endoscope videos diagnosed that the gastric cancer is present in the evaluation test data set) (5)

### Result of Evaluation Test

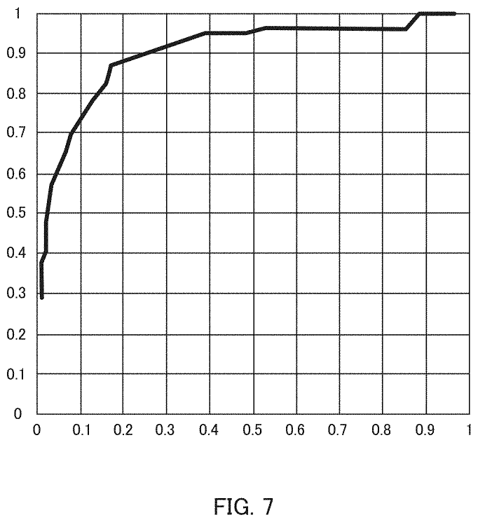

In the evaluation test, the values of the predetermined time, the degree of certainty and the predetermined number with which the correct diagnosis rate of the image diagnosis apparatus is highest were determined. As a result, with a combination of the predetermined time=0.5 seconds, the degree of certainty=0.5, and the predetermined number=3, the correct diagnosis rate (85.1%) of the image diagnosis apparatus was highest. Then, as a result of generation of the ROC curve (see FIG. 7) thereof, AUC was calculated to be 0.8684.

It was confirmed that the condition for 80% or greater of the correct diagnosis rate of the image diagnosis apparatus and AUC greater than 0.8 also includes, in addition to the above-mentioned combination, a combination of the predetermined time=0.1 seconds to 0.5 seconds, the degree of certainty=0.6 and the predetermined number=1, a combination of the predetermined time=0.1 seconds to 0.5 seconds, the degree of certainty=0.5 and the predetermined number=3, a combination of the predetermined time=0.3 seconds to 0.5 seconds, the degree of certainty=0.45 and the predetermined number=5, and a combination of the predetermined time=0.2 seconds, the degree of certainty=0.4 and the predetermined number=5.

Under the condition (the predetermined time=0.5 seconds, the degree of certainty=0.5 and the predetermined number=3) with which the correct diagnosis rate of the image diagnosis apparatus is highest, the correct diagnosis rate, sensitivity, specificity, positive predictive value, and negative predictive value of the image diagnosis apparatus were calculated. Results were the correct diagnosis rate=85.1% (95% CI: 79.0 to 89.6), the sensitivity=87.4% (95% CI: 78.8 to 92.8), the specificity=82.8% (95 % CI: 73.5 to 89.3), the positive predictive value=83.5% (95% CI: 74.6 to 89.7), and the negative predictive value=86.7% (77.8 to 92.4).

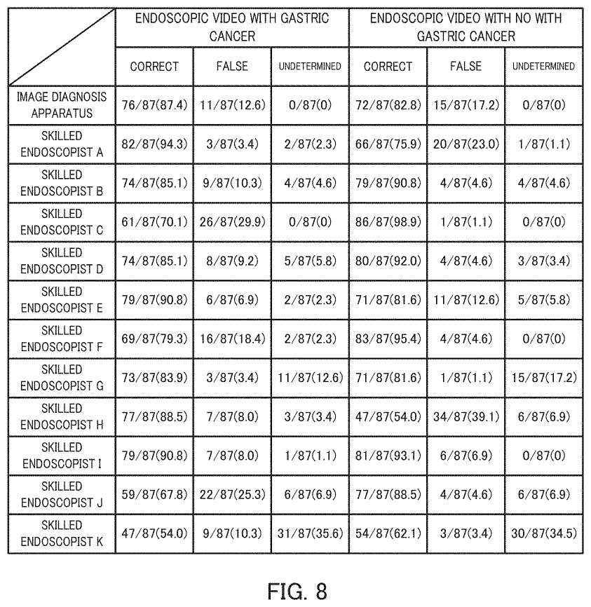

In addition, in the evaluation test, the diagnostic capability of the image diagnosis apparatus and the diagnostic capability of the skilled endoscopist (specialist) were compared with each other. FIG. 8 is a diagram illustrating the numbers of correct diagnoses, false diagnoses and undetermined diagnoses of the image diagnosis apparatus and 11 skilled endoscopists A to K, for an endoscope video where the gastric cancer is present and an endoscope video where the gastric cancer is not present.

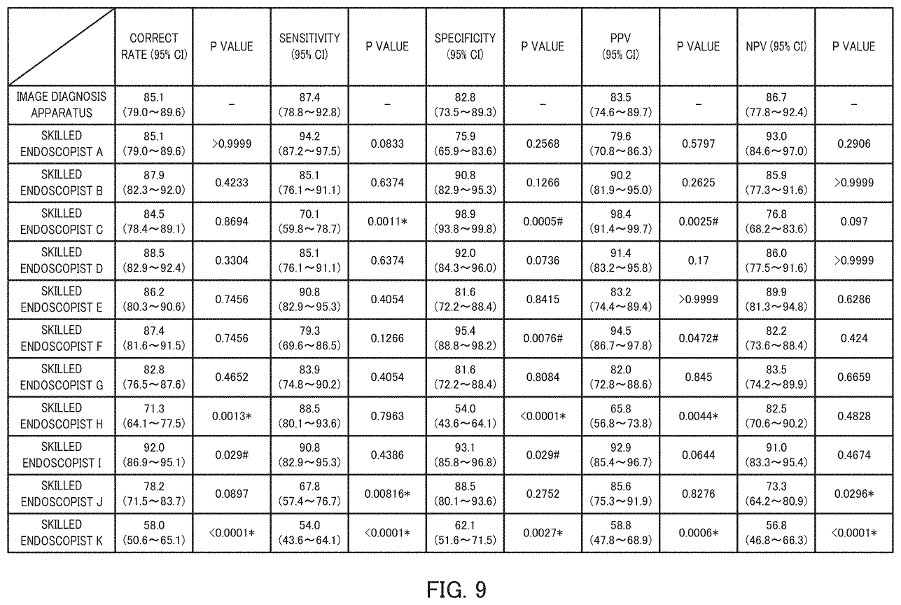

FIG. 9 is a diagram illustrating the correct diagnosis rate, sensitivity, specificity, positive predictive value and negative predictive value of the image diagnosis apparatus and 11 skilled endoscopists A to K. In FIG. 9, the comparison was made by also calculating the 95% confidence interval for the image diagnosis apparatus and the skilled endoscopists A to K. For the comparison between the image diagnosis apparatus and the skilled endoscopists A to K, Mcnemar test was used for the correct diagnosis rate, sensitivity and specificity, while binominal test was used for the positive predictive value and negative predictive value (see the P value of FIG. 9). Here, in each test, statistically significant difference was set to less than 0.05. In this evaluation test, “JMP13” was used as a high-performance interactive tool for visualizing the data and performing statistics analysis.

As illustrated in FIG. 9, in terms of correct diagnosis rate, the image diagnosis apparatus was superior to two skilled endoscopists H and K and significantly inferior to one skilled endoscopist I. In addition, there was no significant difference between the image diagnosis apparatus and eight skilled endoscopists A to G and J.

In terms of sensitivity, the image diagnosis apparatus was significantly superior to three skilled endoscopists C, J and K. In addition, there was no significant difference between the image diagnosis apparatus and eight skilled endoscopists A, B, D to I.

In terms of specificity, the image diagnosis apparatus was significantly superior to two skilled endoscopists H and K, and significantly inferior to three skilled endoscopists C, F, I. In addition, there was no significant difference between the image diagnosis apparatus and six skilled endoscopists A, B, D, E, G and J.

In terms of positive predictive value, the image diagnosis apparatus was significantly superior to two skilled endoscopists H and K, and significantly inferior to two skilled endoscopists C and F. In addition, there was no significant difference between the image diagnosis apparatus and seven skilled endoscopists A, B, D, E, G, I and J.

In terms of negative predictive value, the image diagnosis apparatus was significantly superior to two skilled endoscopists J and K. In addition, there was no significant difference between the image diagnosis apparatus and nine skilled endoscopists A to I.

### Considerations for Evaluation Test

As described above, the values of the predetermined time, the degree of certainty and the predetermined number with which the correct diagnosis rate of the image diagnosis apparatus is highest were determined. As a result, the correct diagnosis rate of the image diagnosis apparatus (85.1%) was highest with the combination of the predetermined time=0.5 seconds, the degree of certainty=0.5 and the predetermined number=3. This means that the optimum diagnosis condition for the diagnosis of gastric cancer with the image diagnosis apparatus is the diagnosis of the presence of the gastric cancer in the endoscope video when three continuous endoscopic images with the degree of certainty of 0.5 or greater are present within 0.5 seconds. That is, in the actual clinical practice, when the gastric cancer can be clearly detected for 0.5 seconds in the endoscope video (ten seconds) (when three endoscopic images with the degree of certainty of 0.5 or greater are continuously present), the gastric cancer can be diagnosed in real time with a high correct diagnosis rate. In addition, even when the degree of certainty is low, the diagnostic capability of the image diagnosis apparatus tends to be maintained by increasing the number of endoscopic images required for diagnosing the presence of the gastric cancer. In the result of evaluation test, the values of the predetermined time, the degree of certainty and the predetermined number with which the correct diagnosis rate of the image diagnosis apparatus is highest are shown, but in the case where the correct diagnosis rate of 70% or greater or 80% or greater is to be maintained, such a diagnostic capability can be achieved with a combination of more broad predetermined time, degree of certainty and predetermined number.

It should be noted that for the actual diagnostic capability of the image diagnosis apparatus, the diagnostic capability of the image diagnosis apparatus and the diagnostic capabilities of 11 skilled endoscopists were compared with each other because it is difficult to perform the evaluation with the image diagnosis apparatus alone. As a result, overall, the image diagnosis apparatus was found to have the same or better diagnostic capability as the skilled endoscopists. The endoscopy is an endoscopy for performing the diagnosis of gastric cancer, and therefore the sensitivity is most important. In the result of the evaluation test, the image diagnosis apparatus was superior to the skilled endoscopist especially in sensitivity. In view of this, the diagnosis of gastric cancer with the image diagnosis apparatus was found to be useful not only for the support (support) of the diagnosis of an endoscopist who have not mastered the diagnosis technique of gastric cancer of ME-NBI, but also for a skilled endoscopist who have mastered the diagnosis technique.

NPL 3 discloses that evaluation of the diagnostic capability of the gastric cancer of a computer-aided diagnosis (CAD) system by using an endoscopic image (still picture image) captured with an NBI combined magnifying endoscope resulted in a correct diagnosis rate of 85.3%, a sensitivity of 95.4%, a specificity of 71.0%, a positive predictive value of 82.3%, and a negative predictive value of 91.7%. In addition, it discloses examples of causes of false positive results include severe atrophic gastritis, localized atrophy, and intestinal epithelialization. In NPL 3, however, no comparison was made between the diagnostic capability of the computer-aided diagnosis system and the diagnostic capability of a skilled endoscopist who have mastered the diagnosis technique of gastric cancer of ME-NBI, and therefore the difficulty of the endoscopic image diagnosis used for evaluating the diagnostic capability is unknown, thus limiting the interpretation of the diagnostic capability of the computer-aided diagnosis system.

In addition, NPL 4 discloses that through consideration similar to that of NPL 3, the image diagnosis apparatus is significantly superior to two skilled endoscopists in sensitivity and negative predictive value. However, because the number of skilled endoscopists compared with the computer-aided diagnosis system is small, that is, the result can be strongly biased by the diagnostic capability of each skilled endoscopist, the difficulty of the endoscopic image diagnosis used for evaluating the diagnostic capability is unknown, thus limiting the interpretation of the diagnostic capability of the computer-aided diagnosis system. In addition, in NPL 4, AUC is also not calculated, and the diagnosis accuracy of the image diagnosis apparatus of the computer-aided diagnosis system is also unknown. Furthermore, in NPLS 3 and 4, still picture images (endoscopic images) are used for the consideration, which is useful for the case where secondary reading of the endoscopic image is performed after the endoscopy; however, because consideration using videos is not performed, it is difficult to be introduced to the actual medical field where the diagnosis of gastric cancer is performed in real time.

NPL 5 discloses that the sensitivity of pick-up diagnosis of gastric cancer was 94.1% in the diagnostic capability of a computer-aided diagnosis system with an endoscope video captured using a typical endoscope. However, in NPL 5, sufficient evaluation cannot be made due to the limitation on the evaluation of the difficulty of the diagnosis of the endoscope video and the interpretation of the diagnostic capability of gastric cancer of the computer-aided diagnosis system, determination of usefulness in hands-on medical care is unknown because of the following points: only the evaluation on the sensitivity is described; the endoscope video captured using an NBI combined magnifying endoscope is not used; the diagnostic capability is not compared between the computer-aided diagnosis system and the skilled endoscopist; and the AUC in the computer-aided diagnosis system is not calculated.

As described above, the known preceding techniques do not perform consideration with a real time video, and therefore the evaluation of the usability and accuracy in the actual clinical practice is insufficient in comparison with the present invention. In contrast, the present invention achieves means for solving the problems, and is superior to the known technology in the following points.
(1) The AUC of the image diagnosis apparatus in the present invention is 0.8684, can be the comprehensive diagnostic capability and the reliability were extremely high as a medical equipment.
(2) In the image diagnosis apparatus of the present invention, which is compared with a large number of skilled endoscopists in terms of the diagnostic capability, the parameter setting and the weighting in CNN are appropriate, and the difficulty of the video evaluation can be properly evaluated. Through the comparison with a large number of skilled medical practitioners, the bias resulting from the comparison with a small number of skilled medical practitioners can be reduced and adjusted. Thus, the computer-aided diagnosis (CAD) system can provide a diagnostic capability performance comparable to or greater than that of the skilled medical practitioners. In addition to the utilization in the actual clinical practice, applicability as education and training system is proved.
(3) In the present invention, an NBI combined magnifying endoscope is used, and thus in comparison with a normal endoscope and an NBI combined non-magnifying endoscope, lesions can be observed in more detail and the diagnostic capability is higher, achieving higher usability in the actual clinical practice.
(4) In the present invention, videos are used instead of still pictures, the diagnosis of gastric cancer can be performed in real time by using the image diagnosis apparatus in the actual clinical practice. In this manner, the task and time for rechecking and determining the still picture after the endoscopy can be eliminated, and the diagnosis of gastric cancer can be promptly supported at the time of endoscopy, thus achieving high excellency in terms of inspection efficiency and cost effectiveness.
(5) The diagnosis using still pictures only evaluates photographed one, and consequently the number of cancers detected at the time of endoscopy is limited. With the video of the present invention, the stomach mucosa can be continuously observed regardless of the capturing timing of the affected area unlike still pictures, and it is very useful in the actual clinical practice for gastric cancer surveillance in terms of the points that cancer detection is assisted in real time during the inspection and that the number of cancers to be detected is not limited.

This application is entitled to and claims the benefit of Japanese Patent Application No. 2020-070848 filed on Apr. 10, 2020, the disclosure each of which including the specification, drawings and abstract is incorporated herein by reference in its entirety.

### INDUSTRIAL APPLICABILITY

The present invention is useful as an image diagnosis apparatus, an image diagnosis method, an image diagnosis program and a learned model that can perform the diagnosis of gastric cancer in real time in gastrointestinal endoscopy using an NBI combined magnifying endoscope.

### REFERENCE SYMBOLS LIST

10 Endoscopic video acquisition section

20 Estimation section

30 Display control section

40 Learning apparatus

100 Image diagnosis apparatus

101 CPU

102 ROM

103 RAM

104 External storage apparatus

105 Communication interface

200 Endoscope capturing apparatus

300 Display apparatus

D1 Endoscope video data

D2 Estimation result data

D3 Determination result image data

D4 Training data

What is claimed is:

1. An image diagnosis apparatus, comprising:
an endoscopic video acquisition section configured to acquire an endoscope video captured in a state where a stomach of a subject is irradiated with narrowband light and the stomach is observed in a magnified manner; and
an estimation section configured to estimate the presence of a gastric cancer in the endoscope video acquired, by using a convolutional neural network, and output an estimation result, the convolutional neural network having been subjected to learning with a gastric cancer image and a non-gastric cancer image as training data.

2. The image diagnosis apparatus according to claim 1,
wherein the estimation section estimates a position of a gastric cancer present in the endoscope video; and
wherein the image diagnosis apparatus further comprises a display control section configured to display the position of the estimated gastric cancer on the endoscope video in a superimposed manner.

3. The image diagnosis apparatus according to claim 2,
wherein the estimation section estimates a degree of certainty of the position of the gastric cancer; and
wherein when the degree of certainty estimated is equal to or greater than a predetermined value, the display control section displays the position of the gastric cancer on the endoscope video in a superimposed manner.

4. The image diagnosis apparatus according to claim 3, wherein when a predetermined number of endoscopic images with the degree of certainty equal to or greater than the predetermined value is continuously present within a predetermined time in the endoscope video, the estimation section estimates that a gastric cancer is present in the endoscope video.

5. The image diagnosis apparatus according to claim 4, wherein the predetermined number becomes greater as the predetermined value becomes smaller.

6. The image diagnosis apparatus according to claim 4, further comprising an alert output control section configured to output an alert when it is estimated that a gastric cancer is present in the endoscope video.

7. An image diagnosis method comprising:
acquiring an endoscope video captured in a state where a stomach of a subject is irradiated with narrowband light and the stomach is observed in a magnified manner; and
estimating the presence of a gastric cancer in the acquired endoscope video by using a convolutional neural network, and outputting an estimation result, the convolutional neural network having been subjected to learning with a gastric cancer image and a non-gastric cancer image as training data.

8. An image diagnosis program configured to cause a program to execute:
an endoscopic video acquisition process of acquiring an endoscope video captured in a state where a stomach of a subject is irradiated with narrowband light and the stomach is observed in a magnified manner; and
an estimation process of estimating the presence of a gastric cancer in the acquired endoscope video by using a convolutional neural network, and outputting an estimation result, the convolutional neural network having been subjected to learning with a gastric cancer image and a non-gastric cancer image as training data.

9. A learned model obtained through learning of a convolutional neural network with a gastric cancer image and a non-gastric cancer image as training data, the learned model being configured to cause a computer to estimate the presence of a gastric cancer in an endoscope video captured in a state where a stomach of a subject is irradiated with narrowband light and the stomach is observed in a magnified manner, and output an estimation result.
